(19) United States
(12) Patent Application Publication
(10) Pub. No.: US 2017/0152212 A1
(43) Pub. Date: Jun. 1, 2017

(54) METHOD FOR PREPARING N,N'-BIS(3-AMINOPROPYL)-1,2-ETHYLENEDIAMINE

(71) Applicant: NAN YA PLASTICS CORPORATION, Taipei (TW)

(72) Inventors: Te-Chao LIAO, Taipei (TW); Jung-Jen CHUANG, Taipei (TW); Zhi-Ming HUANG, Taipei (TW); Shun-Chi CHEN, Taipei (TW); Tzu-Chiang LIN, Taipei (TW)

(21) Appl. No.: 15/355,111

(22) Filed: Nov. 18, 2016

(30) Foreign Application Priority Data
Nov. 26, 2015 (TW) 104139370

(51) Int. Cl. C07C 209/68

(52) U.S. Cl. CPC C07C 209/68

(57) ABSTRACT

A method for preparing N,N′-bis(3-aminopropyl)-1,2-ethylenediamine uses a glycol ether based solution, such as a solution containing dipropylene glycol dimethyl ether (PM) to replace the traditionally used monol-based solvents, and performs hydrogenation in the presence of a Co—Mn—Al catalyst. The method improves the yield of N,N′-bis(3-aminopropyl)-1,2-ethylenediamine to 98.85-99.49% and effectively suppresses generation of by-products.

### BACKGROUND OF THE INVENTION

1. Field of the Invention

The present invention relates to N,N′-bis(3-aminopropyl)-1,2-ethylenediamine, and more particularly to a method for preparing N,N′-bis(3-aminopropyl)-1,2-ethylenediamine with improved yield.

### 2. Description of Related Art

Conventionally, synthesis of N,N′-bis(3-aminopropyl)-1,2-ethylenediamine (hereinafter shortened as BAEDA) is achieved using a monol-based solvent and a metal catalyst containing cobalt or nickel. However, usage of monol-based solvent leads to many by-products that have negative impact on the quality of the final products. As a result, the yield of BAEDA is limited and costly purification systems may be required to refine the final products.

For example, U.S. Pat. No. 5,434,262 discloses a process where BAEDA and ethanol as a solvent perform hydrogenation in the presence of Raney nickel catalyst to get the expected product with a yield of 60%.

As another example, GB 2067191B discloses a method of hydrogenating polynitriles to polyamines using a pelleted cobalt-zinc hydrogenation catalyst.

A further instance is US 2008/0194857A1, which discloses a method of using Raney Cobalt as a catalyst to prepare BAEDA in the presence of isopropanol whose product gave a 98.16% yield as determined by gas chromatography (GC).

Additionally, it is known in the art to use a monol-based solvent (ethanol, isopropanol or methanol) and Raney nickel as a catalyst for hydrogenation. As a result, ethanol and isopropanol are preferred solvents for preparing BAEDA through hydrogenation, which contribute to a yield of 95.80%.

### SUMMARY OF THE PRESENT INVENTION

To break the stereotype that BAEDA is conventionally made in the presence of a monol-based solvent as a solvent in hydrogenation, the present invention discloses a method for preparing BAEDA with high selectivity, which uses dipropylene glycol dimethyl ether (PM) as a solvent and uses a cobalt-manganese-aluminum catalyst (hereinafter referred to as Co—Mn—Al catalyst) to improve the yield of BAEDA to 98.85-99.49% while effectively suppressing generation of by-products.

The disclosed method for preparing BAEDA comprises the following synthesis steps:
a) taking N,N′-bis(2-cyanoethyl)-1,2-ethylenediamine (hereinafter shortened as BCNEDA) as the reactant (the base material);
b) in a high-pressure reactor, adding 1.00-2.00 wt % of a Co—Mn—Al catalyst and 33.21-38.53 wt % of a PM-LiOH solution based on the weight of the base material, wherein the PM-LiOH solution is prepared by dissolving 1 mol % of LiOH based on the BCNEDA reactant into 29-35 wt % of a glycol-ether-based solution based on the total reactant weight, in which the glycol-ether-based solution is prepared by mixing 89-92 wt % of a dipropylene glycol dimethyl ether (PM) solvent and 8-11 wt % of water;
c) under high speed stirring injecting BCNEDA gradually into the reactor using a syringe pump within 3 hours in an environment of 110-130° C. and 700-900 psi;
d) allowing reaction to continuously take place at 120° C.; using gas chromatography (GC) to verify that conversion of BCNEDA reaches 100% and finishing the reaction if so. Analysis of the product of the reaction confirms that the yield of BAEDA is 98.85-99.49%.

### DETAILED DESCRIPTION OF THE INVENTION

The method for preparing BAEDA according to present invention takes a certain amount of BCNEDA as the reactant to perform hydrogenation synthesis in the presence of a PM solution of a specific formula and the final reactant obtained after the reaction improves the yield of BAEDA to 98.85-99.49%.

The disclosed method for preparing BAEDA comprises the following synthesis steps:
a) adding a Co—Mn—Al catalyst with a specific formula into a PM-LiOH solution;
b) transferring the solution of Step a) into a 300 mL agitator-equipped high-pressure reactor that is set with a temperature of 120° C. and a hydrogen pressure of 800 psi, and after the system becomes steady, pumping 93.5 g of BCNEDA into the reactor for hydrogenation; and
c) allowing the reaction to take place continuously at the temperature of 120° C. and the hydrogen pressure of 800 psi, using GC to verify that conversion of BCNEDA reaches 100% and finishing the reaction if so, thereby obtaining a mixture containing BAEDA.

The key technology of the disclosed method for preparing BAEDA relies on using the specially formulated Co—Mn—Al catalyst as the catalyst and the PM-LiOH solution for synthesis, so as to improve the yield of BAEDA to 98.85-99.49% and effectively suppress generation of by-products.

The specially formulated Co—Mn—Al catalyst is uses in an amount of 1.00-2.00 wt % based on the weight of the BCNEDA reactant.

The PM-LiOH solution is prepared by dissolving 1 mol % of LiOH based on the BCNEDA reactant into 29-35 wt % of the glycol ether solution based on the total reactant weight. The glycol-ether-based solution is preferably a mixed solution of a dipropylene glycol dimethyl ether (PM) solvent and water, wherein water takes 8-11 wt % in the mixed solution. Water is added for helping lithium hydroxide to dissolve and distribute across the PM. Lithium hydroxide is used as a catalyst promoter for activating the catalyst and takes 1 mol % of BCNEDA.

While the following examples are herein discussed for further explaining the present invention, the scope of the present invention is not limited thereto.

### EXAMPLE 1

In a 300 mL agitator-equipped high-pressure reactor, 1.000 g of a Co—Mn—Al catalyst (Kawaken OFT-55), 0.065 g of LiOH, 3.230 g of water and 27.752 g of dipropylene glycol dimethyl ether (PM) solution were introduced. In an environment of a constant temperature of 120° C. and a hydrogen pressure of 800 psi, 93.500 g BCNEDA was pumped into the reactor.

After the BCNEDA was pumped in, the mixture was allowed to continue reaction at 120° C. Gas chromatography (hereinafter shortened as GC) was used to confirm that conversion of BCNEDA reached 100%.

After the reaction, a final reaction product was analyzed in terms of composition, and the results are shown in Table 1. The yield of BAEDA is 99.49%.

### EXAMPLE 2

The process is similar to that of Example 1, but had the amount of the Co—Mn—Al catalyst (Kawaken OFT-55) increased to 1.500 g.

After the reaction, a final reaction product was analyzed in terms of composition, and the results are shown in Table 1. The yield of BAEDA is 99.00%.

### EXAMPLE 3

The process is similar to that of Example 1, but a different Co—Mn—Al catalyst (Kawaken OFT-MS) was used.

After the reaction, a final reaction product was analyzed in terms of composition, and the results are shown in Table 1. The yield of BAEDA is 99.15%.

### EXAMPLE 4

The process is similar to that of Example 2, but a different Co—Mn—Al catalyst (Kawaken OFT-MS) was used.

After the reaction, a final reaction product was analyzed in terms of composition, and the results are shown in Table 1. The yield of BAEDA is 98.85%.

### COMPARATIVE EXAMPLE 1

In a 180 mL high-pressure reactor, 1.0700 g of Raney cobalt 2724 catalyst, 0.2520 g of LiOH.H2O, 1.0000 g of H2O, 29.8 g of isopropanol were introduced. In an environment of a constant temperature of 120° C. and a hydrogen pressure of 800 psi, 100.4 g BCNEDA was pumped into the reactor.

After the BCNEDA was pumped in, the mixture was allowed to continue reaction at 120° C. Gas chromatography (hereinafter shortened as GC) was used to confirm that conversion of BCNEDA reached 100%.

After the reaction, a final reaction product was analyzed in terms of composition, and the results are shown in Table 1. The yield of BAEDA is 98.16%.

### COMPARATIVE EXAMPLE 2

In a 300 mL high-pressure reactor, 12.000 g of Raney nickel catalyst, 100.00 g of ammonia anhydrous, 20.000 g of the PM solvent were introduced. In an environment of a constant temperature of 120° C. and a hydrogen pressure of 800 psi, 93.500 g of BCNEDA was pumped into the reactor.

After the BCNEDA was pumped in, the mixture was allowed to continue reaction at 120° C. Gas chromatography (hereinafter shortened as GC) was used to confirm that conversion of BCNEDA reached 100%.

After the reaction, a final reaction product was analyzed in terms of composition, and the results are shown in Table 1. The yield of BAEDA is 85.10%.

### COMPARATIVE EXAMPLE 3

In a 1000 mL high-pressure reactor, 25.000 g of Raney nickel catalyst, 50.00 g of ammonia anhydrous and 20.000 g of methanol were introduced. In an environment of a constant temperature of 65° C. and a hydrogen pressure of 3.5-4.5 MPa, 400 g of BCNEDA that contained 118.383 g of methanol was pumped into the reactor.

After the BCNEDA was pumped in, the mixture was allowed to continue reaction at 55-65° C. Gas chromatography (hereinafter shortened as GC) was used to confirm that conversion of BCNEDA reached 100%.

After the reaction, a final reaction product was analyzed in terms of composition, and the results are shown in Table 1. The yield of BAEDA is 85.80%.

### COMPARATIVE EXAMPLE 4

In a 1000 mL high-pressure reactor, 25.000 g of Raney nickel catalyst, 2.000 g of sodium hydroxide and 20.000 g of ethanol were introduced. In an environment of a constant temperature of 55° C. and a hydrogen pressure of 1.5 MPa, 400 g of BCNEDA that contained 118.383 g of ethanol was pumped into the reactor.

After the BCNEDA was pumped in, the mixture was allowed to continue reaction at a temperature of 60-70° C. and a hydrogen pressure of 1.5-2.0 MPa. Gas chromatography (hereinafter shortened as GC) was used to confirm that conversion of BCNEDA reached 100%.

After the reaction, a final reaction product was analyzed in terms of composition, and the results are shown in Table 1. The yield of BAEDA is 95.80%.

### COMPARATIVE EXAMPLE 5

The process is similar to that of Example 1, but the amount of the Co—Mn—Al catalyst (Kawaken OFT-55) was reduced to 0.500 g.

After the reaction, the product was analyzed in terms of composition, and the results are shown in Table 1. The yield of BAEDA is 79.31%.

### COMPARATIVE EXAMPLE 6

The process is similar to that of Comparative Example 5, but a different Co—Mn—Al catalyst (Kawaken OFT-MS) was used.

After the reaction, the product was analyzed in terms of composition, and the results are shown in Table 1. The yield of BAEDA is 77.43%.

TABLE 1

| | | Examples | | | | Comparative Examples | | | | | |
|---|---|---|---|---|---|---|---|---|---|---|---|
| Components | | 1 | 2 | 3 | 4 | 1 | 2 | 3 | 4 | 5 | 6 |
| BCNEDA(g) | | 93.50 | 93.50 | 93.50 | 93.50 | 100.4 | 93.50 | 400 | 400 | 93.50 | 93.50 |
| Catalyst | Raney Ni¹ (g) | | | | | | 12.000 | 25.000 | 25.000 | | |
| | Raney Co² (g) | | | | | 1.0700 | | | | | |
| | Co—Mn—Al3 (g)) | 1.000 | 1.500 | 1.000 | 1.500 | | | | | 0.500 | 0.500 |
| | | (OFT- | (OFT- | (OFT- | (OFT- | | | | | (OFT- | (OFT- |
| | | 55) | 55) | MS) | MS) | | | | | 55) | MS) |
| Catalyst | LiOH (g) | 0.065 | 0.065 | 0.065 | 0.065 | 0.2520 | | | | 0.065 | 0.065 |
| promoter | NH³ (g) | | | | | | 100.00 | 50.00 | | | |
| | NaOH (g) | | | | | | | | 2.000 | | |
| Solvent | H2O (g) | 3.230 | 3.230 | 3.230 | 3.230 | 1.0000 | | | | 3.230 | 3.230 |
| Alcohol | Methanol (g) | | | | | | 138 | | | | |
| Solvent | Ethanol (g) | | | | | | | 138 | | | |
| | Isopropanol (g) | | | | | 29.8 | | | | | |
| | Dipropylene Glycol | 27.752 | 28.05 | 29.92 | 32.73 | | 20.000 | | | 27.752 | 27.752 |
| | Dimethyl Ether (g) | | | | | | | | | | |

| | | | | | | | | | | |
|---|---|---|---|---|---|---|---|---|---|---|
| Ratio between Catalyst and | 1.07 | 1.60 | 1.07 | 1.60 | 1.07 | 12.83 | 6.25 | 6.25 | 0.53 | 0.53 |
| Reactant (wt %) | | | | | | | | | | |
| Ratio between Solvent and | 33.14 | 33.45 | 35.45 | 38.46 | 30.68 | 21.39 | 34.59 | 34.59 | 33.14 | 33.14 |
| Reactant (wt %)⁴ | | | | | | | | | | |
| Ratio between Dipropylene | | | | | | | | | | |
| Glycol Dimethyl Ether and | 29.68 | 30.0 | 32.0 | 35.0 | — | 21.39 | — | — | 29.68 | 29.68 |
| Reactant (wt %) | | | | | | | | | | |
| BCNEDA's Yield⁵ (%) | 99.49 | 99.00 | 99.15 | 98.85 | 98.16 | 85.10 | 85.80 | 95.80 | 79.31 | 77.43 |

Note:
¹Raney Ni, produced by DUGUSSA Company;
²Raney Co, produced by Grace's Davison;
³Co—Mn—Al, produced by Kawaken Company;
⁴Solvent Ratio (%) = (Water + Alcohols)/BCNEDA
⁵Yield (%) = Conversion (%) × Selectivity (%).

### CONCLUSION

1. As learned from Examples 1-4, in the process of preparing BAEDA, by using a glycol-ether-based solvent such as dipropylene glycol dimethyl ether (PM) of a certain ratio to perform hydrogenation in the presence of a specially formulated Co—Mn—Al catalyst, the reaction was made safe and the yield of BAEDA reached 98.85-99.49%.

2. As demonstrated in Comparative Examples 1-4, in the process of preparing BAEDA, use of a monol-based solvent with Raney Ni or Raney Co as the catalyst for hydrogenation, the yield of BAEDA could only achieve 85.10-98.16%.

3. As learned from Comparative Examples 5-6, in the process of preparing BAEDA, by using a glycol-ether-based solvent such as PM of a certain ratio to perform hydrogenation in the presence of 0.53 wt % of Co—Mn—Al catalyst, the yield of BAEDA was 77.43% and 79.31% respectively. The amount of the catalyst was too low to improve the yield of BAEDA.

What is claimed is:

1. A method for preparing N,N′-bis(3-aminopropyl)-1,2-ethylenediamine, comprising:
a) in a high-pressure reactor, adding 1.00-2.00 wt % of a Co—Mn—Al catalyst and 33.21-38.53 wt % of a dipropylene glycol dimethyl ether-LiOH solution based on a weight of N,N′-bis(2-cyanoethyl)-1,2-ethylenediamine (BCNEDA) as a base material;
b) in an environment of a temperature of 110-130° C. and a pressure of 700-900 psi, gradually pumping BCNEDA into the reactor under high speed stirring using a syringe pump within 3 hours;
c) after pumping, allowing the mixture to continue reaction at 120° C.;
d) using gas chromatography (GC) to verify that conversion of BCNEDA reaches 100% and finishing the reaction; and
e) a yield of N,N′-bis(3-aminopropyl)-1,2-ethylenediamine (BAEDA) ranging between 98.85% and 99.49% is obtained.

2. The method of claim 1, wherein the Co—Mn—Al catalyst is added in an amount of 1.0 wt %.

3. The method of claim 1, wherein N,N′-bis(2-cyanoethyl)-1,2-ethylenediamine is continuously introduced for hydrogenation.

4. The method of claim 1, wherein the pressure for synthesis is 800 psi.

5. The method of claim 1, wherein BCNEDA is gradually pumped into the reactor at the temperature of 120° C.

6. The method of claim 1, wherein the N,N′-bis(2-cyanoethyl)-1,2-ethylenediamine is pumped in over a duration less than three hours.

7. The method of claim 1, wherein the dipropylene glycol dimethyl ether is added in an amount of 33 wt % based on the weight of the BCNEDA reactant.

8. The method of claim 1, wherein the dipropylene glycol dimethyl ether-LiOH solution is prepared by dissolving 1 mol % of LiOH based on the BCNEDA reactant into 29-35 wt % of a glycol-ether-based solution based on the weight of the BCNEDA reactant, and the glycol-ether-based solution is prepared by mixing 89-92 wt % of a dipropylene glycol dimethyl ether (PM) solvent with 8-11 wt % of water.
